(19) United States
(12) Patent Application Publication
(10) Pub. No.: US 2025/0099017 A1
(43) Pub. Date: Mar. 27, 2025

(54) METHODS AND SYSTEMS FOR EARLY DETECTION OF OCULAR AND/OR NEUROLOGICAL CONDITIONS

(71) Applicant: Cedars-Sinai Medical Center, Los Angeles, CA (US)

(72) Inventors: Maya Koronyo, Los Angeles, CA (US); Jonah Doustar, Los Angeles, CA (US); Yosef Koronyo, Los Angeles, CA (US); Keith L. Black, Los Angeles, CA (US)

(21) Appl. No.: 18/580,962

(22) PCT Filed: Jul. 22, 2022
(86) PCT No.: PCT/US2022/074083

§ 371 Date: Jan. 19, 2024

Related U.S. Application Data
(60) Provisional application No. 63/225,279, filed on Jul. 23, 2021.

(51) Int. Cl. A61B 5/00; G06T 7/00

(52) U.S. Cl. CPC A61B 5/4088; A61B 5/4836; A61B 5/4842; A61B 5/489; G06T 7/0012; G06T 2207/30041; G06T 2207/30101

(57) ABSTRACT

The present invention describes systems and method for the detection, diagnosis and monitoring of neurological and/or ocular disease, such as cognitive impairment and Alzheimer's disease. In one example, a method includes differentiating between vein vessels and artery vessels in one or more retinal images of a superior-temporal region of the retina, identifying distal vein branches downstream from a primary vein branch, and quantifying an amount of venular-adjacent amyloid-beta deposits in a venular-adjacent region corresponding to the distal vein branches, wherein the distal vein branches include one or more of secondary veins, secondary vein branches, and tertiary veins. The method further comprises diagnosing the neurological and/or ocular disease based on the amount of venular-adjacent amyloid-beta deposits.

### CROSS-REFERENCE TO RELATED APPLICATIONS

This application claims priority to and the benefit of U.S. Provisional Patent Application No. 63/225,279, filed Jul. 23, 2021, which is hereby incorporated by reference herein in its entirety.

### FIELD OF INVENTION

This invention relates to detection and monitoring of ocular and neurological diseases; and more particularly relates to detection and monitoring of ocular and neurological diseases associated with abnormal amyloidosis, amyloid-beta, and tau pathology, for example, Alzheimer's disease (AD), age-related macular degeneration (AMD), glaucoma, cerebral amyloid angiopathy (CAA), etc.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

The retina is a central nervous system (CNS) tissue that can be visualized through direct non-invasive imaging. Pathological hallmarks of Alzheimer's Disease (AD)—the abnormal deposition of amyloid β-protein (Aβ) and hyperphosphorylated pTau protein aggregates have been identified in the retinas of AD patients, including in early-stage patients with mild cognitive impairment (MCI). In particular, vascular Aβ plaques near and within blood vessels have been identified in the retina of patients. The retinal Aβ deposition may correlate with and may even precede cerebral Aβ deposition. Thus, there is a need for early amyloid deposition detection.

### SUMMARY

Methods and systems are provided for in vivo and non-invasive early detection of Aβ plaques resulting from deposition of Aβ protein in the retina, including at vascular-adjacent areas in the retina. To increase visibility of vascular structures in the retina, some methods use exogenous fluorophores. In some other approaches curcumin, a natural fluorochrome, is administered to the patients to label the retinal Aβ plaques. In any approach, an overall increase in retinal Aβ plaque is correlated with disease indication.

However, the inventors herein have unexpectedly found that the location of the plaque deposits and their association with specific vessels show greater correlation with the neurological and/or ocular disease. In particular, the inventors have unexpectedly found that arterial-associated plaque deposits were overall more abundant in both cognitively impaired patients and cognitively normal patients than venular-associated plaque deposits. However, when patients were separated by diagnostic group and cognitive performance, the venular-associated plaques had an increased quantity in MCI/AD patients compared against their cognitively normal counterparts.

Accordingly, in one example, a method for evaluating a neurological and/or ocular health condition of a patient comprises: acquiring one or more retinal images; identifying arterial-associated plaques and venular-associated plaques in the one or more retinal images; determining an arterial-associated plaque count of the arterial-associated plaques; determining a venular-associated plaque count of the venular-associated plaques; and detecting a neurological and/or ocular disease based on the arterial-associated plaque count and the venular-associated plaque count.

In this way, by differentiating the Aβ plaques that are associated with the veins from the Aβ plaques that are associated with the arteries, accuracy in early detection of one or more neurological and/or ocular diseases is increased. The venular-associated plaques provide a unique retinal biomarker that correlates with neurodegenerative/ocular diseases. Accordingly, the methods and systems described herein provide great improvement in accuracy and efficiency in early and in vivo evaluation of neurological and/or ocular health condition.

### BRIEF DESCRIPTION OF THE FIGURES

Example embodiments are illustrated in referenced figures. It is intended that the embodiments and figures disclosed herein are to be considered illustrative rather than restrictive.

FIG. 1 shows is a schematic diagram illustrating a retinal imaging system, according to an embodiment of the disclosure;

FIG. 2 shows a flowchart illustrating an example method for assessing neurological and/or ocular health condition associated with amyloidosis, according to an embodiment of the disclosure;

FIG. 3 shows a flowchart illustrating an example method for classifying retinal vascular architecture for amyloid-beta plaque detection, according to an embodiment of the disclosure;

FIG. 4 shows a flowchart illustrating an example method for determining neurological and/ocular health condition based on vascular-adjacent plaque deposition, according to an embodiment of the disclosure;

FIG. 5 shows an example retinal image including example vein architecture labelling, venular-adjacent area segmentation, and curcumin-labelled plaques;

FIG. 6 shows the retinal image of FIG. 5 including example artery architecture labelling, arterial-adjacent area segmentation, and curcumin-labelled plaques;

FIGS. 7A-7F show graphs illustrating example counting of all plaque positive signals that fall within the boundaries of the vessel adjacent area and separated by vessel type (near-venous or near-arterial) and location (primary, primary branch, secondary, secondary branch, or tertiary);

FIGS. 8A-8C show graphs depicting retinal amyloid plaque count in venular-adjacent region for individuals stratified by Clinical Dementia Rating (CDR) scores;

FIGS. 9A-9C show graphs depicting retinal amyloid plaque count in venular-adjacent region for individuals stratified by diagnostic group (NC=normal control; and MCI/AD=mild cognitive impairment/Alzheimer's disease);

FIGS. 10A-10C show graphs depicting retinal amyloid plaque counts in venular-adjacent regions for individuals stratified by the Montreal Cognitive Assessment (MOCA) score of 26; and

FIGS. 11A-11C show ratio between venular-adjacent and arterial-adjacent plaque counts (V/A ratio) with respect to total vessels (FIG. 11A), secondary vessels (FIG. 11B), and secondary branch vessels (FIG. 11C).

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art may recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

### Overview

Alzheimer's disease (AD) and associated dementia are estimated to afflict 50 million people worldwide, a number projected to triple by year 2050. This age-dependent epidemic is a major concern for the aging population, with an incidence that rises sharply after 65 years of age, affecting roughly 50% of individuals aged 85 and older. While currently there is no cure, with early diagnosis, the progression of the disease may be slowed.

Although AD has been historically perceived as a brain disorder, recent studies indicate that AD also manifests in the eye with mounting evidence of abnormalities in the retina, a sensory extension of the brain. Particularly, the hallmark pathological signs of AD, amyloid β-protein (AB) and neurofibrillary tangles (NFTs) comprised of hyperphosphorylated pTau protein, which have long been described in the brain, have also been identified in the retina. There is a growing number of reports that Aβ deposits and pTau were discovered in the retinas of AD patients at various stages, in stark contrast to non-AD controls. Previous approaches have shown that retinal Aβ deposits in AD and MCI patients correlating with brain Aβ plaque burden. As the only CNS tissue not shielded by bone, the retina, offers unique access to study pathological changes in the brain.

Further, some approaches have shown that perivascular and vascular Aβ deposits were also identified in retinas from MCI and AD patients. Accordingly, current approaches involve quantification of general increase in Aβ plaques.

However, the inventors have recognized that classification of vascular-associated plaques is a key factor in providing more accurate diagnosis of neurological and/or ocular diseases associated with amyloidosis and Aβ deposition. In particular, the inventors have identified that similar quantities of arterial-associated plaques can be detected in both cognitively normal and cognitively impaired patients, while venular-associated plaques are significantly increased in patients showing cognitive decline/impairment. Accordingly, the venular-associated plaques provide a unique retinal biomarker that may be used for more accurate disease diagnosis.

In this way, by classifying and quantifying arterial-associated plaques and venular-associated plaques, accuracy in in vivo detection of Aβ plaques is greatly increased.

Further, in some examples, veins and arteries are further sub-classified, into corresponding secondary and tertiary vessels. Further, association of Aβ plaques with the secondary and tertiary arteries and veins may be determined, and neurological and/or ocular disease diagnosis may be based on taking into account the association of Aβ plaques with the secondary and tertiary vessels. Alternatively, in some examples, diagnosis focus may be targeted to Aβ plaque association with the secondary and/or tertiary arteries and veins.

In further examples, in addition to secondary and tertiary arteries and veins, secondary and tertiary branches of the corresponding vessels are classified, where the branches are protrusions from the corresponding vessels (e.g., secondary artery branch protrudes from secondary artery, secondary vein branch protrudes from secondary vein, tertiary artery branch protrudes from tertiary artery, and tertiary vein protrudes from tertiary vein). Accordingly, detection of Aβ plaques associated with different vessel branches may be used for diagnosis of the neurological and/or ocular diseases. As used herein, the term “plaque” refers to an Aβ plaque unless otherwise noted.

### Systems and Methods

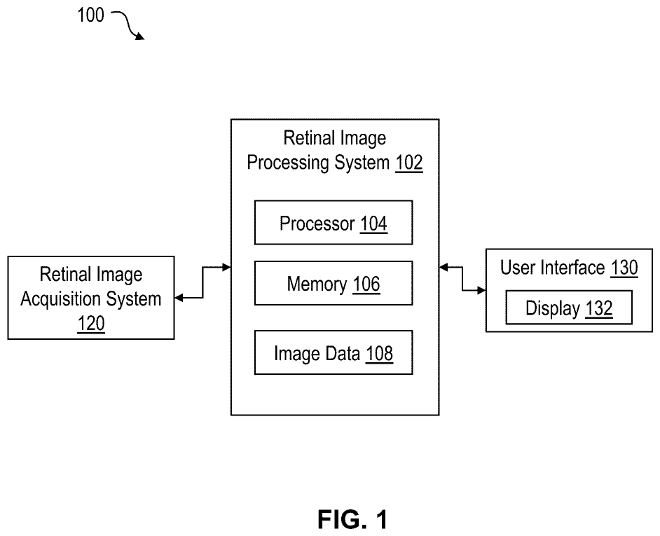

FIG. 1 illustrates a retinal imaging system 100. In some embodiments, as shown, a retinal image acquisition system 120 is communicatively coupled to a retinal image processing system 102. The retinal image acquisition system 120 may acquire one or more retinal images and subsequently transmit the retinal image data (via a wired and/or a wireless connection) to the retinal image processing system 102 for image processing and/or reconstruction, and subsequently, one or more of retinal vessel classification, vessel-adjacent area segmentation, vessel-associated Aβ plaque detection, quantification of vessel-associated Aβ plaque, and evaluation of neurological and/or ocular health condition.

In some examples, the retinal image data acquired via the retinal image acquisition system 120 may be wirelessly transmitted to the integrated processing unit and processed therein, and/or transmitted the retinal image processing system 102 communicatively coupled to the retinal image acquisition system 120.

In one example, the retinal imaging system 100 may be a confocal scanning ophthalmoscope. The confocal scanning ophthalmoscope may include one or more laser light sources. The confocal scanning ophthalmoscope may further include scanning optics and/or adaptive optics for acquiring retinal images at different focal planes.

In one non-limiting example, a patient may be administered curcumin (e.g., Longvida curcumin) as a contrast agent for Aβ plaque staining according to a desired curcumin loading protocol (e.g., 2-day, 3-day, 4-day, 5-day, 6-day, 7-day, 8-day, 9-day, 10-day, etc.). At the time of acquisition, following ocular dilation, the confocal scanning ophthalmoscope may be used to obtain retinal images using blue light for excitation of curcumin emission to obtain images of the retina. In one example, the excitation LED emitting peak wavelengths of light at 452 nanometers may be used, and further a barrier filter may be used to collect fluorescent emissions of curcumin greater than or equal to 500 nm. Further, in one example, the camera field of view may be 60 degrees (H)×55 degrees (V), and the nominal optical resolution on the retina may be 17 μm.

In other examples, the retinal imaging system 100 may include imaging systems such as an optical coherence tomography (OCT) system or an optical coherence tomography angiography (OCT-A) system, either of which may be configured to detect vessels and Aβ plaque (with or without staining) in the superior-temporal retina at about 60° field of view.

In various embodiments, the retinal imaging system 100 may be any of adaptive optics, optical coherence tomography/OCT-angiography system, color fundus photography system, fluorescein angiography system, indocyanine green angiography system, scanning laser ophthalmoscopy system, optical coherence tomography system, spectral-OCT, confocal ophthalmoscopy system, or retinal hyperspectral imaging system.

In some embodiments, the retinal image processing system 102 is disposed at a device (e.g., edge device, server, etc.) communicably coupled to the retinal image acquisition system 120 via wired and/or wireless connections. In some embodiments, the retinal image processing system 102 is disposed at a separate device (e.g., a workstation) which can receive image data from the retinal image acquisition system 120 or from a storage device which stores the image data acquired by the retinal image acquisition system 120. The retinal image processing system 102 may comprise at least one processor 104, and a user interface 130 which may include a user input device (not shown), and a display device 132. User input device may comprise one or more of a touchscreen, a keyboard, a mouse, a trackpad, a motion sensing camera, or other device configured to enable a user to interact with and manipulate data within the retinal imaging system 100.

The at least one processor 104 is configured to execute machine readable instructions stored in non-transitory memory 106. The processor 104 may be single core or multi-core, and the programs executed thereon may be configured for parallel or distributed processing. In some embodiments, the processor 104 may optionally include individual components that are distributed throughout two or more devices, which may be remotely located and/or configured for coordinated processing. In some embodiments, one or more aspects of the processor 104 may be virtualized and executed by remotely-accessible networked computing devices configured in a cloud computing configuration. According to other embodiments, the processor 104 may include other electronic components capable of carrying out processing functions, such as a digital signal processor, a field-programmable gate array (FPGA), or a graphic board. According to other embodiments, the processor 104 may include multiple electronic components capable of carrying out processing functions. For example, the processor 104 may include two or more electronic components selected from a list of electronic components including: a central processor, a digital signal processor, a field-programmable gate array, and a graphic board.

In still further embodiments the processor 104 may be configured as a graphical processing unit (GPU) including parallel computing architecture and parallel processing capabilities. However, it will be appreciated that a trained neural network model as described herein for perioperative risk evaluation may be implemented in a processor that does not have GPU processing capabilities.

Non-transitory memory 106 may store a retinal image post-processing module (not shown) and retinal image data 112. In some examples, the retinal image post-processing module may store a neural network model comprising a plurality convolutional layers or a machine learning model. In some examples, the retinal image post-processing module may include instructions that when executed may cause the processor to classify and segment retinal vascular architecture, including vein architecture and artery architecture as further discussed below.

Further, the retinal image post-processing module may include instructions that when executed may cause the processor to segment vessel adjacent area in one or more classified vessels. Furthermore, the retinal image processing module may be configured to detect Aβ plaque within a desired region of interest (e.g., vessel-adjacent area) and quantify Aβ plaque with the desired region of interest.

Further, the retinal image post-processing module may include instructions that when executed may cause the processor to generate a composite image from a plurality of images. As a non-limiting example, a set of images (e.g., 18 images) of the superior retina may be acquired for each eye, including six images at each of the three different focal planes (autofocus and ±2 machine diopters) to accommodate focus variability and eye curvature. The set of retinal images may be processed screened for image quality (including focus, contrast, variation in illumination, eye motion, obstruction, and proper fixation), and the eight highest quality images may be selected for further processing. These eight images may be aligned and combined to reduce noise and further processed to reduce background variability and to maximize dynamic range. An example retinal imaging and processing method is provided by Dumitrascu et al (Dumitrascu, Oana M et al. “Sectoral segmentation of retinal amyloid imaging in subjects with cognitive decline.” Alzheimer's & dementia (Amsterdam, Netherlands) vol. 12,1 e12109. 28 Sep. 2020, doi: 10.1002/dad2.12109), which is incorporated herein by reference in its entirety.

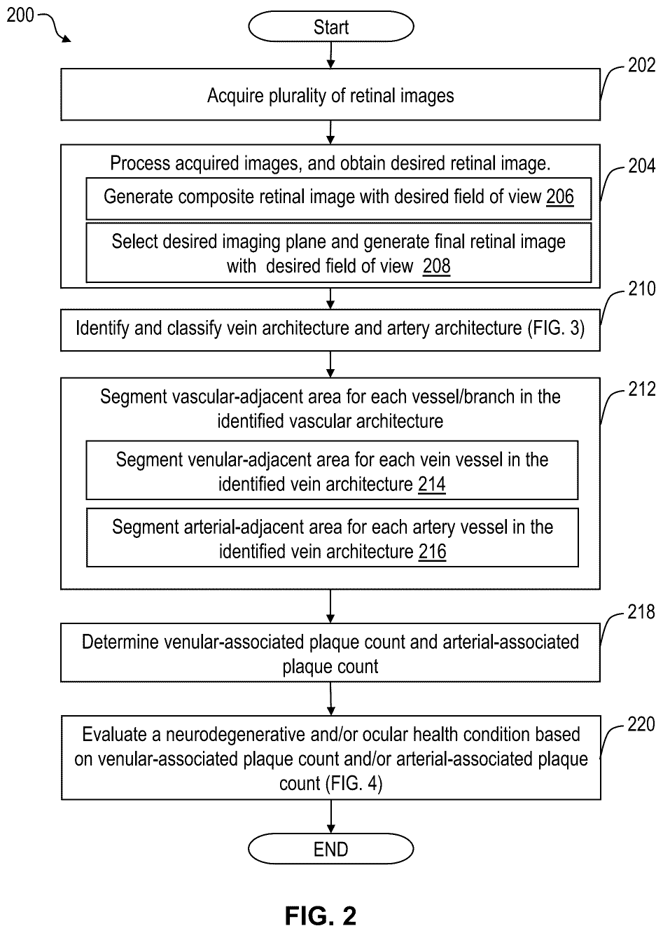

The retinal image processing module may further include instructions for implementing an algorithm to receive retinal image data of a patient acquired from retinal image acquisition system and output a corresponding neurological and/or ocular disease classification (e.g., severe, mild, intermediate; presence or absence of cognitive impairment, etc.) for mortality and/or one or more of cardiovascular adverse conditions based on vessel-associated plaque identification and quantification. In some examples, the algorithm may be a trained neural network model or a trained machine learning model. For example, the retinal image processing module may store instructions that, when executed by processor 104, cause the retinal image processing system 102 to conduct one or more of the steps of method 200, 300, and 400 (FIGS. 2, 3, and 4) discussed in more detail below.

In some examples, non-transitory memory 106 may also store training and inference modules that comprises instructions for validating and testing new data with the trained neural network or machine learning models. Non-transitory memory 106 further stores the retinal image data 112. Retinal image data includes for example, plurality of retinal images acquired by a retinal image processing system. In some embodiments, the retinal image data 112 may include a plurality of training sets, each comprising a plurality of retinal images (which may be labelled for supervised training, for example).

In some embodiments, the non-transitory memory 106 may include components disposed at two or more devices, which may be remotely located and/or configured for coordinated processing. In some embodiments, one or more aspects of the non-transitory memory 106 may include remotely-accessible networked storage devices configured in a cloud computing configuration.

The display device 132 may include one or more display devices utilizing virtually any type of technology. In some embodiments, display device 132 may comprise a computer monitor, and may display unprocessed and processed retinal images. Display device 132 may be combined with processor 104, non-transitory memory 106, and/or user input device in a shared enclosure, or may be peripheral display devices and may comprise a monitor, touchscreen, projector, or other display device known in the art, which may enable a user to view retinal images, and/or interact with various data stored in non-transitory memory 106.

It should be understood that retinal imaging system 100 shown in FIG. 1 is for illustration, not for limitation. Another appropriate retinal imaging system may include more, fewer, or different components.

FIG. 2 illustrates a flowchart of an example method 200 for evaluating a neurodegenerative and/or ocular health condition using a retinal imaging system, such as the retinal imaging system 100 of FIG. 1. Method 200, and the other methods described herein may be implemented by a computational model stored in non-transitory memory, such as non-transitory memory 106 of the retinal image processing system 102, an edge device connected to the image processing system, a cloud in communication with the retinal image processing system, or any appropriate combination thereof.

At step 202, the method 200 includes acquiring one or more retinal images. The retinal images may be acquired via a retinal image acquisition system. An example retinal image acquisition system may be configured to acquire in vivo retinal images with at least 60° view and configured to image superior-temporal region of the retinal. Further, the retinal image acquisition system may be configured to detect Aβ plaque and detect retinal vessels. As non-limiting examples, an optical coherence tomography (OCT) or optical coherence tomography angiography (OCT-A) or a confocal scanning ophthalmoscope or scanning laser ophthalmoscope, may be used. It will be appreciated that the above examples are provided for illustration, and any retinal imaging system that is configured for in vivo retinal imaging of the retina, including Aβ plaques and blood vessels, with a desired field of view can be implemented without departing from the scope of the disclosure.

Next, at step 204, the method 200 includes processing at least one of the one or more acquired images to obtain one or more desired retinal images. Processing acquired images may include conversion of raw retinal images, and applying one or more of thresholding, normalization, and filtering to improving signal to noise ratio. Accordingly, a controlled and uniform image-processing analysis to improve signal and reduce noise. Further, processing acquired images may include determining and/or setting a spot identification threshold to enable detection of the plaques.

In some examples, a quality control operation may be performed as part of processing the acquired images. Accordingly, the acquired images may be screened for image quality before performing additional processing. In some examples, screening for image quality may be based on focus, contrast, variation in illumination, eye motion, obstruction, and proper fixation. The quality control operation may include selecting a set of high quality images based on one or more of the above parameters in the screening for image quality. Upon selecting the set of highest quality images, the set of high quality images may be aligned and combined to reduce noise and further processed to reduce background variability and to maximize dynamic range.

In some examples, a composite image may be generated from the set of high quality images (indicated at sub-step 206). In another example, a desired imaging plane that meets a plurality of quality control measures may be selected (indicated at sub-step 208). In some other examples, a three dimensional retinal image may be generated from the plurality of images.

Upon obtaining the desired retinal image (after processing), a common region of interest having a desired field of view (e.g., 50, 60, 70, 80, 90, 100, 110, 120, 130, 140, 150, 160, 170, 180, or any value between 50 and 180 degrees) may be selected. Depending on the desired retinal image and acquisition parameters, the ROI field of view may be adjusted. For example, for a three-dimensional retinal image, the ROI field of view may be greater than 180 degrees. As one non-limiting example, the common region of interest (ROI) may have a field of view of 50 degrees positioned on the image center, using fovea and optic nerve head centers as reference points to correct for eye rotation, with a zone around the fovea and optic nerve head masked.

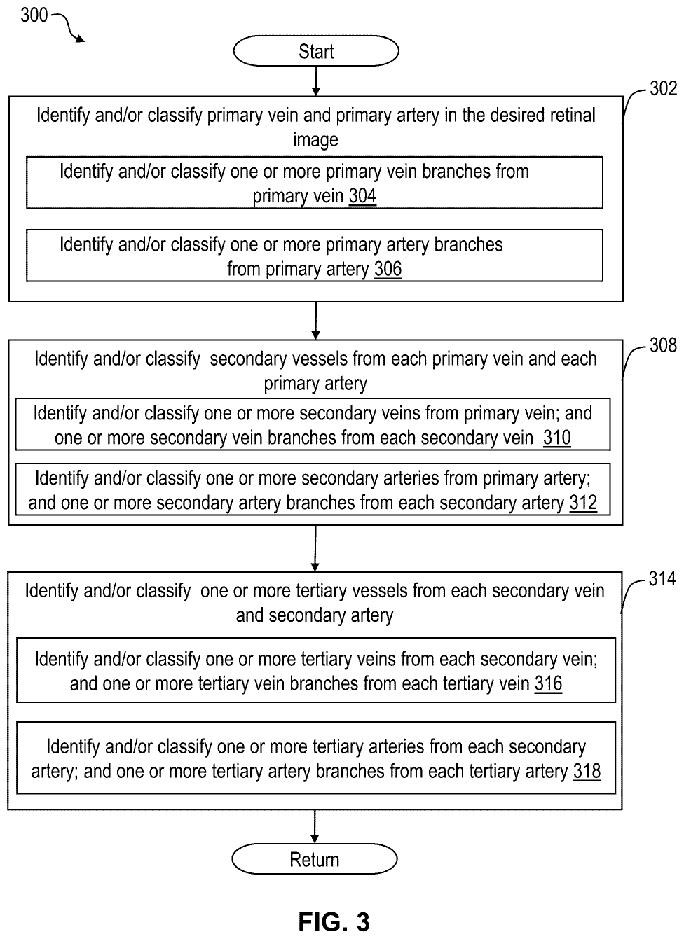

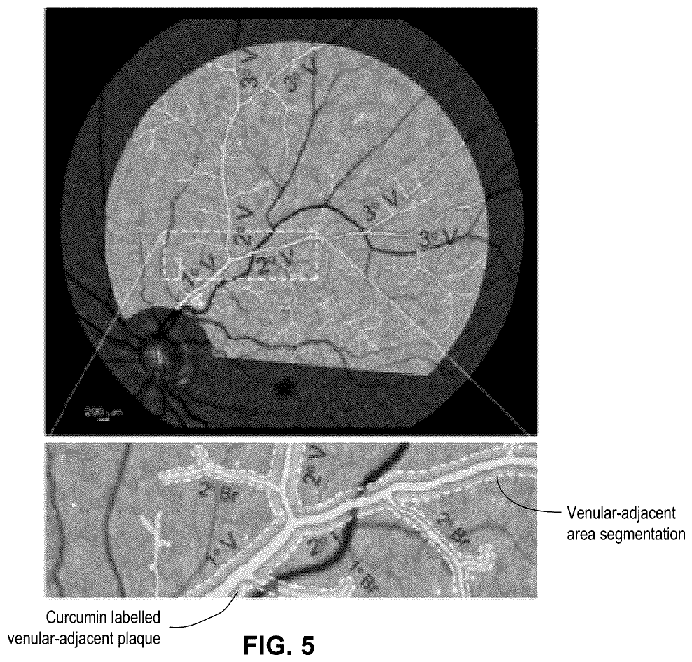

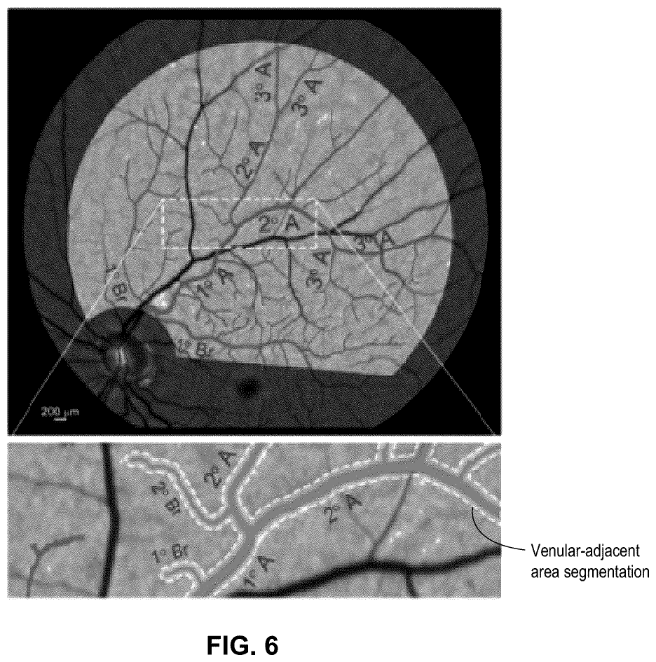

At step 210, the method 200 includes identifying and classifying retinal vessel architecture in the desired retinal image(s). Details of identifying and classifying retinal vessel architecture is discussed below with respect to FIG. 3, which illustrates a flow hart of a method 300 for identifying and classifying retinal vessel architecture according to an embodiment of the disclosure (e.g., method 300 shows the individual steps of step 210 of method 200). The method 300 will be described with respect to FIGS. 5 and 6.

At step 302, the method 300 includes identifying and/or classifying primary vein and primary artery in the desired retinal image. The superior-temporal region of the retina includes a single primary vessel for each vein and artery architecture, where a diameter of the primary vein is greater than a diameter of the primary artery. That is, within the visual field of these superior-temporal retinal images, there is a single main vein (primary vein) and a single main artery (primary artery). These are shown in FIGS. 5 (vein architecture) and 6 (artery architecture), and referred to as 1° V and 1° A respectively in FIGS. 5 and 6. Protruding smaller diameter vessels from each of the primary vein and primary artery are primary vein branches and primary artery branches (1° Br in FIGS. 5 and 6). Accordingly, in some examples, step 302 includes identifying and/or classifying primary vein branches (sub-step 304), and/or identifying and/or classifying primary artery branches (sub-step 306).

At step 308, the method 300 includes identifying and/or classifying secondary vessels from each primary vein and each primary artery. This includes, identifying and/or classifying secondary veins (2° V at FIG. 5) from each primary vein and identifying and/or classifying secondary artery (2° A) from each primary artery. That is, the primary vessel extends until it splits into two equal diameter vessels referred to as secondary(2°) veins/arteries. In some examples, step 308 of the method 300 includes sub-step 310 and/or sub-step 312. At sub-step 310, the method 300 includes identifying and/or classifying one or more secondary vein branches (2° Br at FIG. 5) from each secondary vein. At sub-step 312, the method 300 includes identifying and/or classifying one or more one or more secondary artery branches (2° Br at FIG. 6) from each secondary artery.

At step 314, the method 300 includes identifying and/or classifying tertiary vessels from each secondary vein and each secondary artery. This includes, identifying and/or classifying tertiary veins (3° V at FIG. 5) from each secondary vein and identifying and/or classifying tertiary arteries (3° A) from each secondary artery. That is, the secondary vessels extend until they each split into two equal diameter vessels referred to as tertiary (3°) veins/arteries. In some examples, step 314 of the method 300 includes sub-step 316 and/or sub-step 318. At sub-step 316, the method 300 includes identifying and/or classifying one or more tertiary vein branches (3° Br at FIG. 5) from each tertiary vein. At sub-step 316, the method 300 includes identifying and/or classifying one or more tertiary artery branches (3° Br at FIG. 6) from each tertiary artery.

In this classification scheme, tertiary vessels and their branches will have equal diameters, therefore all vessels following the second split are grouped as tertiary. Thus, based on one or more of diameters of vessels and branching topology, the vessels may be classified as primary, secondary, or tertiary, and further sub-classified as primary, secondary, or tertiary branches (that is, branches of the primary, secondary, and tertiary). Further, during the classification, the vein architecture is differentiated from the artery architecture based on a primary vein diameter and the venular vessels branching from the primary vein, and a primary artery diameter and arterial vessels branching from the primary artery.

Upon identifying and/or classifying the vein architecture and the artery architecture, and the vessels in each of the vein architecture and the artery architecture, the method 300 returns to step 212 at FIG. 2. At step 212, the method 200 includes segmenting vascular-adjacent area for each vessel/branch in the identified vascular architecture. In some examples, step 212 includes sub-step 214 and sub-step 216. At sub-step 214, the method 200 includes identifying and segmenting venular-adjacent area for each vein vessel in the identified and/or classified vein architecture. At sub-step 216, the method 200 includes identifying and segmenting arterial-adjacent area for each artery vessel in the identified and/or classified artery architecture.

In one example, the vascular-adjacent area may be based on diameter of each vessel. Accordingly, in one example, for a given vessel, the vascular adjacent area may track a contour of the vessel and a total vascular adjacent area may be a number of times greater than the vessel area and may include the vessel area. In various examples, the number may range from 1 to 5. An example outline of the vascular-adjacent area is shown at FIGS. 5 and 6 in dashed lines in the corresponding enlarged portions.

In some examples, the vascular-adjacent area may be adjusted changed according to corresponding vessel diameter such that a total area between the vessels and the area adjacent boundary is the same for all vessels. Further, in some examples, the vascular-adjacent area may be adjusted such that there is no overlap or minimal overlap between venular-adjacent areas and arterial-adjacent areas.

In one non-limiting example, starting within the visual field and closest to the optic disc, for each primary vessel (that is, for each primary vein and primary artery) the diameter of the primary vessel may be measured at pre-set intervals along its length, and the average diameter may be calculated. Further, an area corresponding to a number of times of the diameter of the vessel (e.g., 1×,1.5×, 2×, 2.5×, 3×, 3.5×, 4×, 4.5×, 5×, 5.5× etc.) may be calculated to determine the vascular-adjacent area. Similarly, vascular-adjacent area may be determined for all vessels (veins, arteries) in the corresponding vein architectures.

Next, at step 218, the method 200 includes identifying venular-associated plaques and arterial-associated plaques. This includes, counting all plaque positive signals that fall within the boundaries of the vessel adjacent area. Further, the plaque counts may be separated by vessel type (near-venous or near-arterial) and location (primary, primary branch, secondary, secondary branch, or tertiary)

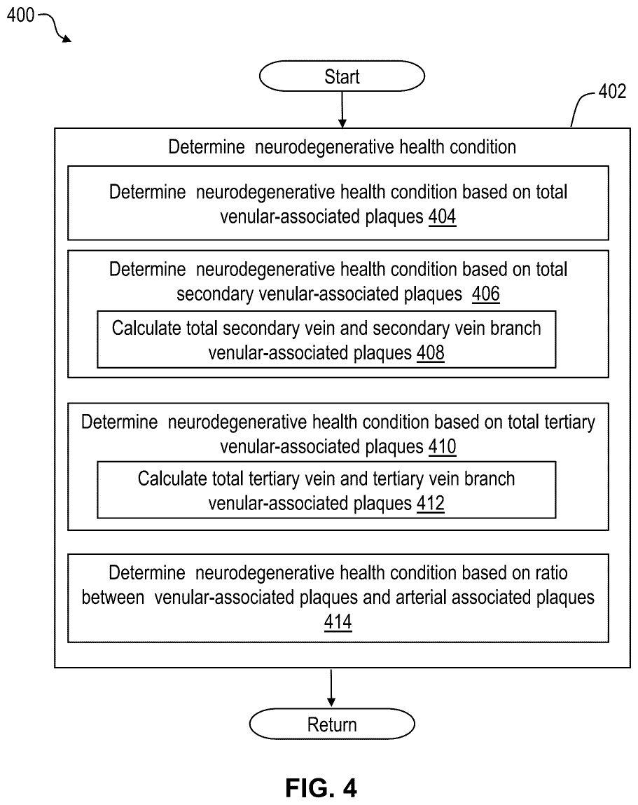

Next, at step 220, the method 200 includes evaluating a neurodegenerative and/or ocular health condition based on venular-associated plaque count and/or arterial-associated plaque count. Details of step 220 are discussed below with respect to FIG. 4, which illustrates a flowchart of a method 400 for evaluating a neurological and/or ocular health condition according to venular-associated and/or arterial-associated plaque count (e.g., the method 400 shows the individual steps of steps 220 of the method 200).

Step 402 of method 400 includes determining a neurodegenerative health condition of a patient. In some examples, step 402 includes sub-step 404. Sub-step 404 of the method 400 includes determining the neurodegenerative health condition based on the amount venular-associated associated plaques. In one example, the amount of venular-associated plaques within a threshold venular-adjacent area may be determined, and the health condition may be indicated in response to the amount of venular-associated plaques being greater than a threshold value. For example, for a given area, when the amount of venular-associated plaques is greater than the threshold, a neurological and/or ocular disease associated with amyloidosis may be indicated. Accordingly, in some examples, when greater retinal imaging area is considered, the threshold may increase. The threshold can be pre-determined. The amount of plaques can be quantified in a variety of different ways. In some cases, the amount of plaques is the number of distinct plaques. In some cases, the amount of plaques is an area and/or volume of plaques. In some cases, the amount of plaques is a density of plaques.

In some examples, step 402 includes sub-step 406. Sub-step 406 of the method 400 includes determining the neurodegenerative health condition based on the amount of secondary venular-associated plaques (e.g., plaques associated with secondary vein vessels and/or secondary vein branches). In some examples, sub-step 406 includes sub-step 408, which includes calculating an amount of secondary vein venular-associated plaques and an amount of secondary vein branch venular-associated plaques. Thus, in some examples, for a given area, when the amount of secondary venular-associated plaques is greater than a secondary venular-associated plaque threshold, a neurological and/or ocular disease associated with amyloidosis may be indicated.

In some examples, step 402 includes sub-step 410. Sub-step 410 of the method 400 includes determining the neurodegenerative health condition based on the amount of tertiary venular-associated plaques (e.g., plaques associated with tertiary vein vessels and/or tertiary vein branches). In some examples, sub-step 410 includes sub-step 412, which includes calculating the amount of tertiary vein venular-associated plaques and the amount of tertiary vein branch venular-associated plaques. Thus, in some examples, for a given area, when the amount of tertiary venular-associated plaques is greater than a tertiary-associated plaque threshold, a neurological and/or ocular disease associated with amyloidosis may be indicated.

In another example, the neurodegenerative health condition may be based on the amount of secondary and tertiary venular-associated plaques. This includes calculating the amount of venular-associated plaques that are associated with secondary vein vessels, tertiary vein vessels, secondary vein branches, and tertiary vein branches. Thus, in some examples, for a given area, when the amount of secondary and tertiary venular-associated plaques is greater than a combined plaque threshold, a neurological and/or ocular disease associated with amyloidosis may be indicated.

In another example, the neurodegenerative health condition may be based on a number of plaques per threshold vascular and perivascular area, where the threshold vascular and perivascular area includes one or more secondary veins, secondary vein branches, tertiary veins, and tertiary vein branches. In one example, the primary veins and primary vein branches may be excluded, and/or arterial-adjacent plaques may be excluded.

In one example, the amount of venular-associated plaques may be determined, and responsive to the amount of venular-associated plaques being greater than a threshold number, the health condition may be indicated based on secondary and/or tertiary venular-associated plaques greater than corresponding thresholds.

In some examples, a plaque density per unit volume for venular-adjacent plaques may be used for indicating a neurological and/or ocular health condition. For example, when 3-dimensional retinal image is evaluated, venular-associated plaque density may be utilized for determining the neurological and/or ocular health condition.

In some examples, vascular-adjacent plaques that are common to both venular-adjacent areas and arterial-adjacent areas may be identified and excluded from the venular-adjacent plaque count.

In some examples, step 402 includes sub-step 414, which includes determining the neurodegenerative health condition based on a ratio between venular-associated plaques and arterial-associated plaques.

In some examples, areas of increased venular-associated plaques on the retinal images can be highlighted. For example, retinal images may be shown on a display device (such as display device 132), and areas of increased venular-associated plaques can be highlighted on the display device. Moreover, an indication of the vein architecture in the retinal images can also be shown on the display device, as can an indication of the segmentation of the venular-adjacent regions in the retinal images.

In this way, by identifying and differentiating venular-associated plaques from arterial-associated plaques, and evaluating a neurological and/or ocular health condition based on venular-associated plaque deposits, accuracy in diagnosis is improved.

Further, the inventors have identified that by focusing on the venular-associated plaques in the superior temporal region of the retina, accuracy in diagnosis is improved. Furthermore, the inventors have identified that distal vein vessel formations following the first major split of the primary vein were subject to the most significant accumulation of these vessel adjacent plaques in disease, and by targeted analysis of venular-associated plaques in the distal vein formations increases accuracy in diagnosis. Furthermore, as the targeted area is narrower, efficiency is improved. Furthermore, a number of false positives is reduced. Furthermore, the methods and systems described herein can be efficiency implemented for early diagnosis of neurological and/or ocular diseases associated with amyloid beta deposition. In this way, the methods and systems described herein provide great improvement in neurodegenerative disease diagnosis.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

FIG. 5 shows an example fluorescent retinal image including labelled vein architecture, venular-adjacent area segmented by dotted lines, and curcumin labelled plaques (yellow). FIG. 6 shows the corresponding artery architecture in the example image of FIG. 5. Similar to FIG. 5, FIG. 6 shows labelled artery architecture, arterial-adjacent area, and curcumin labelled plaques.

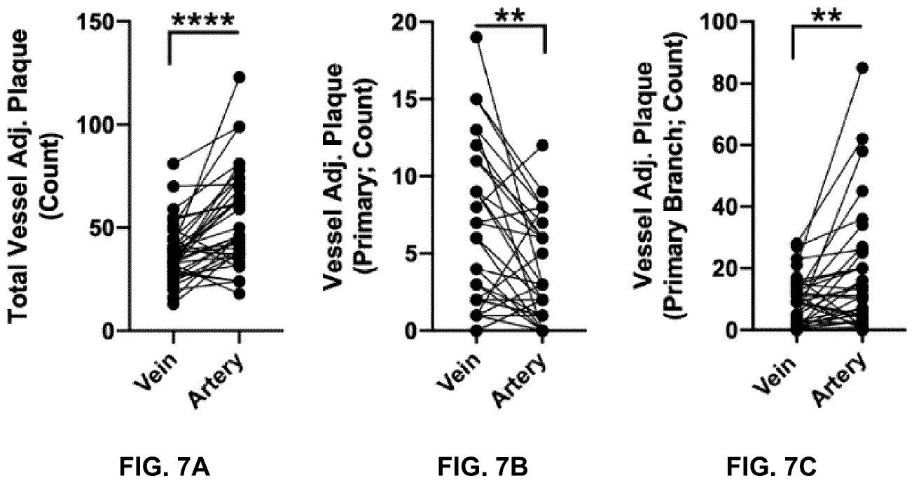

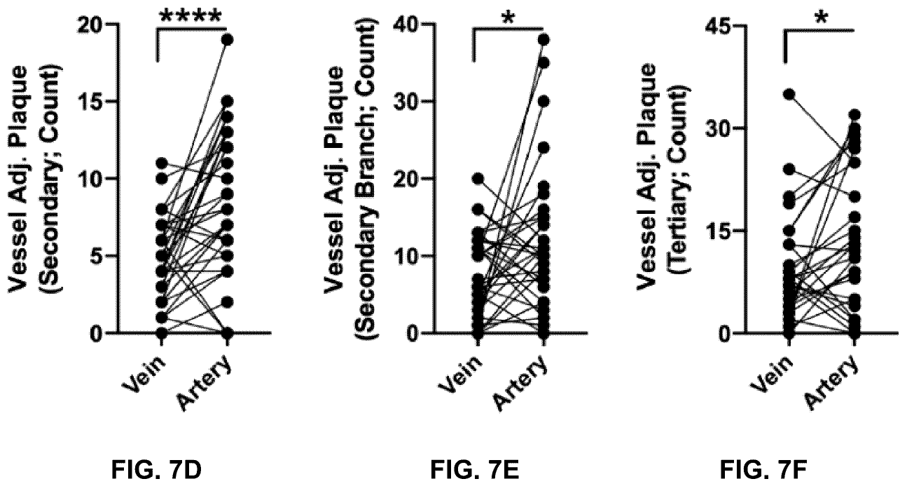

FIG. 7A shows a graph depicting total vessel adjacent plaque distribution between arteries and veins.

FIGS. 7B and 7C show graphs depicting distribution of vessel-adjacent plaques with respect to primary vessels and primary vessel branches respectively.

FIGS. 7D and 7E show graphs depicting distribution of vessel-adjacent plaques with respect to secondary vessels and secondary vessel branches respectively.

FIG. 7F shows a graph depicting vessel adjacent plaque distribution in the tertiary veins and arteries.

The examples shown at FIGS. 7A-7F are for illustrating example counting of all plaque positive signals that fall within the boundaries of the vessel adjacent area and separated by vessel type (near-venous or near-arterial) and location (primary, primary branch, secondary, secondary branch, or tertiary).

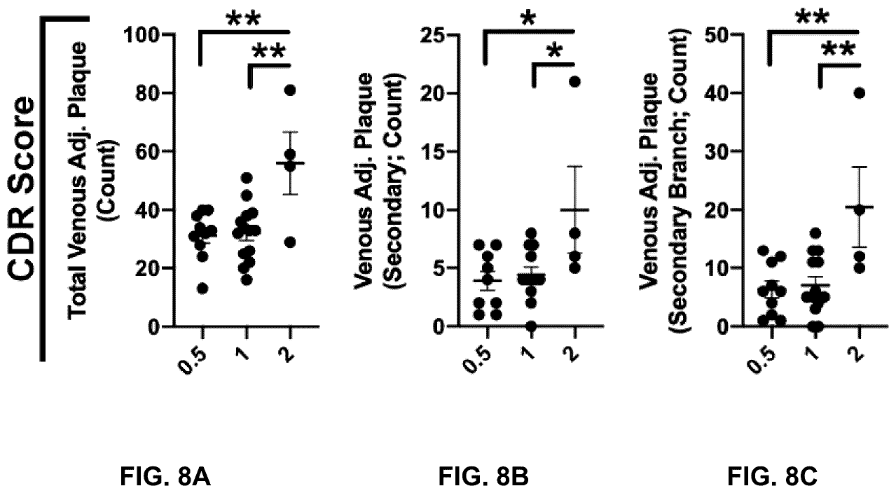

Turning next to FIGS. 8A-8C, the figures shows retinal amyloid plaque count in venular-adjacent region for individuals stratified by Clinical Dementia Rating (CDR) scores. As shown, total venular-adjacent plaques, total secondary venular-adjacent plaques, and total secondary branch venular-adjacent plaques increase with increase in CDR rating, and thus can be utilized to evaluate a neurodegenerative health condition based on amyloid-beta plaque deposition associated with vein architecture.

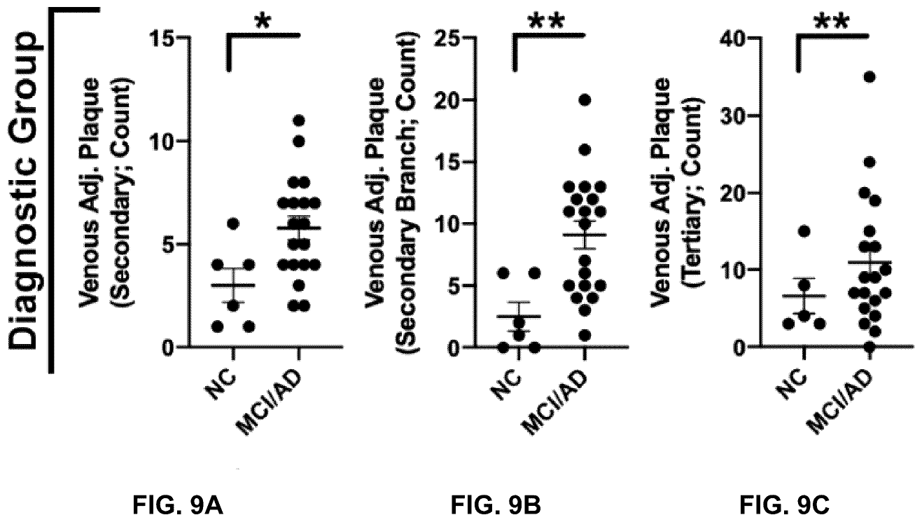

FIGS. 9A-9C show retinal amyloid plaque count in venular-adjacent region for individuals stratified by diagnostic group (NC=normal control; and MCI/AD=mild cognitive impairment/Alzheimer's disease). As shown, total venular-adjacent plaques, total secondary venular-adjacent plaques, and total secondary branch venular-adjacent plaques are greater in MCI/AD subjects compared to subjects with normal cognitive function (NC). Accordingly, venular-adjacent plaque count can be utilized to evaluate neurodegenerative disease conditions based on amyloid-beta plaque deposition associated with vein architecture.

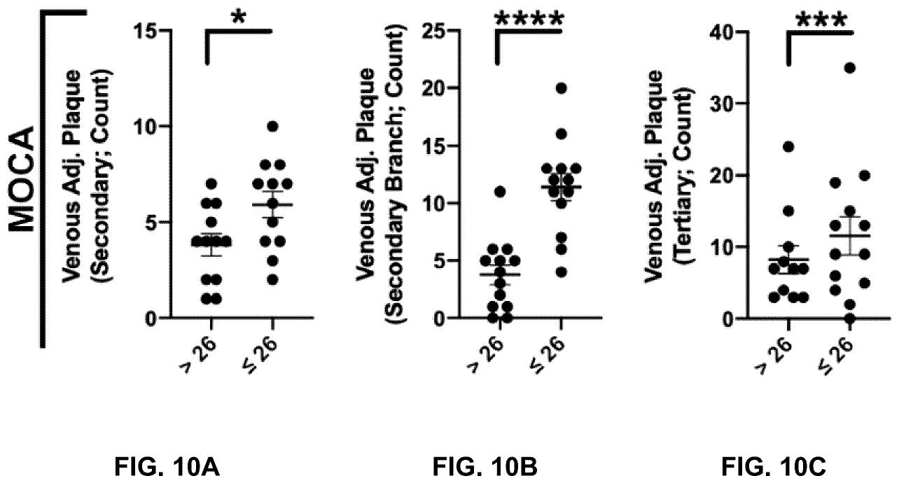

FIGS. 10A-10C show retinal amyloid plaque counts in venular-adjacent regions for individuals stratified by the Montreal Cognitive Assessment (MOCA) score of 26. As shown, total secondary venular-adjacent plaques, total secondary branch venular-adjacent plaques, and total tertiary venular-adjacent plaques are increased in subjects with MOCA score less than 26.

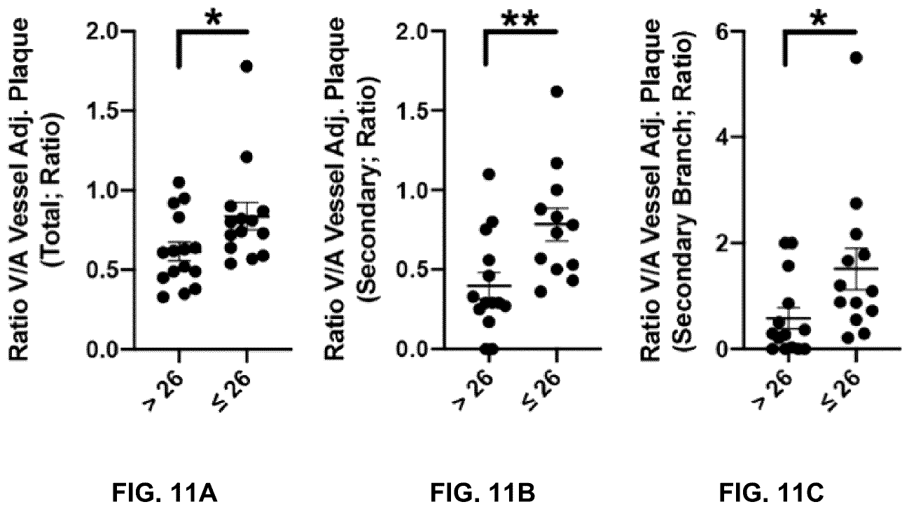

FIGS. 11A-11C show ratio between venular-adjacent and arterial-adjacent plaque counts (V/A ratio) with respect to total vessels (FIG. 11A), secondary vessels (FIG. 11B), and secondary branch vessels (FIG. 11C). As shown, V/A ratios are greater in subjects with MOCA score less than 26.

In view of the above, venular-associated plaque count in venular-adjacent regions, as well as V/A ratios can be used to assess neurodegenerative health conditions.

Accordingly, one or more of venular-adjacent plaque count, primary venular-adjacent plaque count, secondary venular-adjacent plaque count, secondary-branch venular-adjacent plaque count, tertiary venular-adjacent plaque count, and various V/A ratios (total V/A ratio, V/A ratio associated with primary vessels, V/A ratio associated with secondary vessels, V/A ratios associated with secondary branch vessels, V/A ratio associated with tertiary vessels) may be used to evaluate neurological and/or ocular diseases associated with amyloid-beta deposition.

Example neurological and/or ocular diseases associated with amyloid-beta deposition include, but not limited to Alzheimer's Disease (AD), mild cognitive impairment (MCI), glaucoma, and age-related macular degeneration.

In various embodiments, the method further comprises administering a mild cognitive impairment (MCI) or Alzheimer's disease therapy when cognitive impairment is diagnosed in the subject. Therapies to treat MCI or Alzheimer's disease include but are not limited to cholinesterase inhibitors such as donepezil (Aricept), galantamine (Razadyne) and rivastigmine (Exelon), and memantine.

Further, in some examples, Aβ deposits may be quantified in order to determine whether the subject has pathological hallmarks of AD and MCI, as well as a degree of progression of the disease.

Furthermore, in some examples, when the subject is undergoing immunomodulation treatments, reduction in Aβ deposits may be monitored over a duration of the treatment.

Administrations of the various compounds, agents and compositions described herein in accordance with various embodiments of the invention can be any administration pathway known in the art, including but not limited to intravenous, intraocular, intraretinal, subcutaneous, aerosol, nasal, oral, transmucosal, transdermal or parenteral. “Transdermal” administration may be accomplished using a topical cream or ointment or by means of a transdermal patch. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compounds, agents and compositions may be in the form of solutions or suspensions for infusion or for injection, or as lyophilized powders. Via the enteral route, the compounds, agents and compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compounds, agents and compositions may be in the form of solutions or suspensions for infusion or for injection. Via the topical route, the compounds, agents and compositions may be formulated for administration to the skin and mucous membranes and are in the form of ointments, creams, milks, salves, powders, impregnated pads, solutions, gels, sprays, lotions or suspensions. They can also be in the form of microspheres or nanospheres or lipid vesicles or polymer vesicles or polymer patches and hydrogels allowing controlled release. These topical-route compositions can be either in anhydrous form or in aqueous form depending on the clinical indication. Via the ocular route, they may be in the form of eye drops.

In some examples, following in vivo analysis, in test subjects such as mice, ex vivo analysis may be performed (and in case of human, post-mortem analysis). For ex-vivo analysis, certain antibodies or compounds are labelled. The ex vivo label refers to a composition capable of producing a detectable signal indicative of the presence of a target. Suitable labels, for example, for the anti-LRP-1 antibodies anti-PDGFR-β antibodies, and anti-Aβ compounds, include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods and devices described herein. For example, peptides can be labeled with a detectable tag which can be detected using an antibody specific to the label.

Example fluorophores and fluorescent labeling reagents include, but are not limited to, fluorescein, Hydroxycoumarin, Succinimidyl ester, Aminocoumarin, Methoxycoumarin, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, Peridinin chlorphyll protein, TruRed (PerCP-Cy5.5 conjugate), FluorX, Fluoresceinisothyocyanate (FITC), FITC-dextran (2000kD), BODIPY-FL, TRITC, X-Rhodamine (XRITC), Lissamine Rhodamine B, Texas Red, Texas Red-dextran (3kD), Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5 or Cy7.

### Computer & Hardware Implementation of Disclosure

It should initially be understood that the disclosure herein may be implemented with any type of hardware and/or software, and may be a pre-programmed general purpose computing device. For example, the system may be implemented using a server, a personal computer, a portable computer, a thin client, or any suitable device or devices. The disclosure and/or components thereof may be a single device at a single location, or multiple devices at a single, or multiple, locations that are connected together using any appropriate communication protocols over any communication medium such as electric cable, fiber optic cable, or in a wireless manner.

It should also be noted that the disclosure is illustrated and discussed herein as having a plurality of modules which perform particular functions. It should be understood that these modules are merely schematically illustrated based on their function for clarity purposes only, and do not necessary represent specific hardware or software. In this regard, these modules may be hardware and/or software implemented to substantially perform the particular functions discussed. Moreover, the modules may be combined together within the disclosure, or divided into additional modules based on the particular function desired. Thus, the disclosure should not be construed to limit the present invention, but merely be understood to illustrate one example implementation thereof.

The computing system can include clients and servers. A client and server are generally remote from each other and typically interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

Implementations of the subject matter and the operations described in this specification can be implemented in digital electronic circuitry, or in computer software, firmware, or hardware, including the structures disclosed in this specification and their structural equivalents, or in combinations of one or more of them. Implementations of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially-generated propagated signal. The computer storage medium can also be, or be included in, one or more separate physical components or media (e.g., multiple CDs, disks, or other storage devices).

The operations described in this specification can be implemented as operations performed by a “control system” on data stored on one or more computer-readable storage devices or received from other sources.

The term “control system” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application-specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it may be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It may be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It may be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method for diagnosing a neurodegenerative disease in a patient, the method comprising:
acquiring one or more retinal images of a retina of the patient;
identifying venular-adjacent amyloid-beta plaques in one or more venular-adjacent regions of the retina;
calculating an amount of the venular-adjacent amyloid beta plaques; and
diagnosing the neurodegenerative disease based at least in part on the amount of venular-adjacent amyloid-beta plaques.

2. The method of claim 1, wherein diagnosing the neurodegenerative disease comprises:
identifying an amount of arterial-adjacent amyloid beta plaques in one or more arterial-adjacent regions of the retina;
calculating an amount of the arterial-adjacent amyloid beta plaques; and
calculating a ratio between the amounts of venular-adjacent plaques and arterial-adjacent plaques.

3. The method of claim 1, wherein diagnosing the neurodegenerative disease comprises differentiating venular-adjacent amyloid-beta plaques from arterial-associated amyloid-beta plaques.

4. The method of claim 1, wherein identifying venular-adjacent amyloid-beta plaques comprises (i) identifying plaques that fall within the boundaries of a threshold area adjacent to the veins, (ii) identifying a vein architecture of the retina and classifying the vein architecture, or (iii) both (i) and (ii).

5. (canceled)

6. The method of claim 4, wherein identifying the vein architecture comprises identifying (i) one or more primary vein vessels, (ii) one or more primary vein branches, (iii) one or more secondary vein vessels, (iv) one or more secondary vein branches, (v) one or more tertiary vein vessels, or (vi) any combination of (i)-(v).

7. The method of claim 6, wherein the identification of the one or more primary vein branches, the one or more secondary vein vessels, the one or more secondary vein branches, and the one or more tertiary vein vessels is based at least in part on a diameter of the one or more primary vein vessels and a branching of the one or more primary vein vessels.

8. The method of claim 4, wherein the one or more venular-adjacent regions includes areas adjacent to but not within the one or more primary vein vessels, the one or more primary vein branches, the one or more secondary vein vessels, the one or more secondary vein branches, the one or more tertiary vein vessels, or any combination thereof.

9. The method of claim 1, wherein the one or more retinal images include images of a superior-temporal region of the retina.

10. The method of claim 1, wherein the amount of venular-adjacent amyloid-beta plaques includes (i) a density of the amyloid-beta plaques within a threshold volume of venular-adjacent region, (ii) a total number of the amyloid-beta plaques within a threshold area of the venular-adjacent region, or (iii) both (i) and (ii).

11-13. (canceled)

14. The method of claim 1, wherein diagnosing the neurodegenerative disease according to the amount of venular-adjacent amyloid-beta plaques includes determining if the amount of venular-adjacent amyloid-beta plaques is greater than a threshold amount, the threshold amount being (i) based on an amount of arterial-adjacent amyloid beta plaques, (ii) a predetermined threshold amount, or (iii) both (i) and (ii).

15-16. (Canceled)

17. The method of claim 1, further comprising administering a treatment to the patient responsive to diagnosis of the neurodegenerative disease.

18. The method of claim 1, further comprising monitoring a disease progression in the patient based on change in the amount of venular-associated amyloid-beta plaques over time.

19. The method of claim 1, further comprising highlighting an area of increased venular-associated amyloid-beta plaques on the one or more retinal images. 20-21. (Canceled)

22. A method for diagnosing a neurological and/or ocular health condition of a patient, the method comprising:
acquiring one or more retinal images of a superior temporal region of a retina of the patient;
calculating an amount of amyloid-beta plaques in a venular-adjacent region of the retina of the patient; and
diagnosing the neurological and/or ocular health condition based on the amount of amyloid-beta plaques in the venular-adjacent region.

23. A retinal imaging system for assessing a retina of a patient, the system comprising:
a retinal image acquisition system; and
a retinal image processing system including a processor and a non-transitory memory device, the retinal image processing system coupled to the retinal image acquisition system;
wherein the non-transitory memory device includes instructions that when executed by the processor, cause the processor to:
acquire, via the retinal image acquisition system, one or more retinal images of the retina of the patient;
differentiate between vein vessels and artery vessels in the one or more retinal images;
identify (i) venular-adjacent amyloid-beta plaques associated with one or more vein vessels, (ii) arterial-adjacent amyloid-beta plaques associated with one or more artery vessels, or (iii) both (i) and (ii); and
evaluate a neurological and/or ocular health condition based on (i) the venular-adjacent amyloid-beta plaques, (ii) the arterial-adjacent amyloid-beta plaques, or (iii) both (i) and (ii).

24. The retinal imaging system of claim 23, wherein evaluating neurological and/or ocular health condition based on the venular-adjacent amyloid-beta plaques, (ii) the arterial-adjacent amyloid-beta plaques, or (iii) both (i) and (ii) comprises:
determining a venular-adjacent amyloid-beta plaque count based on the venular-adjacent amyloid-beta plaques associated with the one or more vein vessel; and
diagnosing a neurological and/or ocular disease based on the venular-adjacent amyloid-beta plaque count.

25. (canceled)

26. The retinal imaging system of claim 23, wherein the one or more vein vessels include (i) one or more primary vein vessels, (ii) one or more primary vein branches, (iii) one or more secondary vein vessels, (iv) one or more secondary vein branches, (v) one or more tertiary vein vessels, or (vi) any combination of (i)-(v).

27. The retinal imaging system of claim 23, wherein evaluating neurological and/or ocular health condition based on venular-adjacent amyloid-beta plaques and/or arterial-adjacent amyloid-beta plaques comprises:
determining a venular-adjacent amyloid-beta plaque count and an arterial-adjacent amyloid-beta plaque count; and
diagnosing a neurological and/or ocular disease based on the venular-adjacent amyloid-beta plaque count and the arterial-adjacent amyloid-beta plaque count.

28. The retinal imaging system of claim 27, wherein:
the venular-adjacent amyloid-beta plaque count is based on venular-adjacent amyloid-beta plaques associated with the one or more vein vessels; and
the arterial-adjacent amyloid-beta plaque count is based on arterial-adjacent amyloid-beta plaques associated with the one or more artery vessels.

29. The retinal imaging system of claim 28, wherein the one or more vein vessels includes one or more primary vein vessels, one or more primary vein branches, one or more secondary vein vessels, one or more secondary vein branches, one or more tertiary vein vessels, or any combination thereof, and wherein the one or more artery vessels includes one or more primary artery vessels, one or more primary artery branches, one or more secondary artery vessels, one or more secondary artery branches, one or more tertiary artery vessels, or any combination thereof.

30-32. (canceled)
